(19) United States
(12) Patent Application Publication
(10) Pub. No.: US 2017/0333304 A1
(43) Pub. Date: Nov. 23, 2017

(54) Hydrogel Particles, Compositions, and Methods

(71) Applicant: Massachusetts Institute of Technology, Cambridge, MA (US)

(72) Inventors: Natalie Artzi, Brookline, MA (US); Yi Zhang, Cambridge, MA (US); Nuria Oliva Jorge, Somerville, MA (US); Joao Conde, Lisbon (PT)

(21) Appl. No.: 15/599,998

(22) Filed: May 19, 2017

Related U.S. Application Data
(60) Provisional application No. 62/339,434, filed on May 20, 2016.

(51) Int. Cl. A61K 8/18; A61K 8/73; A61K 8/04; A61K 8/02; A61K 8/46; A61K 8/42; C07D 407/10; A61K 31/33

(52) U.S. Cl. CPC A61K 8/18; C07D 407/10; A61K 8/73; A61K 31/33; A61K 8/0241; A61K 8/46; A61K 8/42; A61K 8/042; A61K 2121/00

(57) ABSTRACT

Hydrogel particles are provided that include a crosslinked polymer and a drug conjugated to the crosslinked polymer. The hydrogel particles may include a pH sensitive moiety and a redox sensitive moiety. The hydrogel particles also may be dispersed in a host hydrogel. Kits and methods of drug delivery and treatment also are provided.

### CROSS-REFERENCE TO RELATED APPLICATIONS

This application claims priority to U.S. Provisional Patent Application No. 62/339,434, filed May 20, 2016, which is incorporated herein by reference.

### BACKGROUND

Designing drug delivery devices and compositions for cancer therapy can be a complex endeavor, due at least in part to the fact that some anti-cancer therapeutics benefit from being protected by a carrier and/or highly targeted to a tumor site. Most known devices for cancer treatment belong to two categories: nanoparticles that can be administered intravenously, and hydrogels that can be implanted to provide local release. Both of these systems suffer from one or more disadvantages.

Nanoparticles provide decent protection for therapeutics, and surface modifications can increase the circulation time of nanoparticles. However, the percentage of nanoparticles that eventually arrives at a target site usually is still low. Systemic administration typically requires higher doses of therapeutics, therefore increasing the toxicity caused by particles accumulated at the liver, spleen, or kidneys.

Hydrogels can be implanted or in situ crosslinked at a target site. Therefore, hydrogels may permit the local delivery of therapeutics. However, the loading quantity and/or homogeneity of hydrophobic drugs may be limited, and/or the release of therapeutics typically relies on passive diffusion, which makes it difficult to achieve a controlled and/or sustained release of a therapeutic, particularly small molecule therapeutics, like most chemotherapy drugs.

There remains a need for biocompatible and/or bioabsorbable devices and/or compositions that can deliver one or more therapeutics to a target site, such as a tumor, in a manner that is selective, sustainable, controllable, effective, or a combination thereof.

### BRIEF SUMMARY

Provided here are hydrogel particles and compositions that include one or more hydrogel particles dispersed in a host hydrogel that can controllably and/or selectively deliver one or more therapeutics to a target site, such as a tumor.

In one aspect, compositions are provided that include a hydrogel particle. The hydrogel particle may include (i) a crosslinked polymer that includes a crosslinker, and (ii) a drug conjugated to the crosslinked polymer by a covalent bond that is pH sensitive or redox sensitive. The crosslinker of the crosslinked polymer may include a moiety that is redox sensitive when the covalent bond is pH sensitive, or pH sensitive when the covalent bond is redox sensitive. In one embodiment, the moiety that is redox sensitive comprises a disulfide moiety. In another embodiment, the moiety that is pH sensitive comprises an imine moiety. In a further embodiment, the covalent bond is pH sensitive, and the crosslinker comprises the moiety that is redox sensitive.

In another aspect, methods of treating cancer in a patient also are provided. The methods may include administering to the patient an effective amount of a composition provided herein that is or includes hydrogel particles. The compositions may be applied locally to one or more tumors in a patient's body. Also provided are methods for local delivery of a drug to a biological tissue, which may include applying to a biological tissue a composition, as provided herein, that includes one or more hydrogel particles dispersed in a host hydrogel, and permitting the hydrogel particle(s) to diffuse from the composition into the biological tissue.

In a further aspect, methods of delivering a drug to biological tissue also are provided. The methods may include providing a first solution that includes a first polymer component including a first polymer having one or more aldehydes; providing a second solution including at least one of (i) a dendrimer having at least two branches with one or more surface groups, wherein about 25% to 100% of the surface groups include at least one primary or secondary amine, and (ii) a second polymer component including a second polymer having one or more amines; combining the first and second solutions together to produce a hydrogel composite; and contacting one or more biological tissues with the hydrogel composite, wherein at least one of the first solution and the second solution includes a composition, as provided herein, that is or includes one or more hydrogel particles.

In yet another aspect, kits for making a hydrogel composite are provided. The kits, in embodiments, include a first part that includes a first solution including a first polymer component that includes a first polymer having one or more aldehydes; and a second part that includes a second solution including at least one of (i) a dendrimer having at least two branches with one or more surface groups, wherein about 25% to 100% of the surface groups include at least one primary or secondary amine, and (ii) a second polymer component including a second polymer having one or more amines, wherein at least one of the first solution and the second solution includes a composition, as provided herein, that is or includes one or more hydrogel particles. In one embodiment, the kit includes a syringe, wherein the first solution and the second solution are stored in the syringe.

### BRIEF DESCRIPTION OF THE DRAWINGS

FIG. 1 depicts one embodiment of a kit containing the components of a hydrogel composite or composition.

FIG. 2 depicts the results of an MTT assay of embodiments of hydrogel particles with poly(lactic-co-glycolic acid) (PLGA) particles as control.

FIG. 3 depicts the cumulative release of doxorubicin from an embodiment of a dextran:doxorubicin conjugate at different pHs.

FIG. 4 depicts the cumulative release of doxorubicin from an embodiment of a dextran:doxorubicin conjugate in the presence or absence of glutathione.

FIG. 5 depicts the cumulative release of doxorubicin from an embodiment of a dextran:doxorubicin conjugate in the presence of glutathione at different pHs.

FIG. 6 depicts the release of an embodiment of hydrogel particles from a host hydrogel under conditions that mimic a healthy environment and a cancerous environment.

FIG. 7 depicts the cumulative release of doxorubicin from embodiments of hydrogel compositions that include an embodiment of a redox sensitive hydrogel particle or a non-redox sensitive hydrogel particle under conditions that mimic a healthy environment and a cancerous environment.

FIG. 8 depicts the therapeutic efficacy of an embodiment of hydrogel nanoparticles (NPs) without doxorubicin and an embodiment of hydrogel nanoparticles with doxorubicin for 3T3 fibroblasts and MDA-MB-231 breast cancer cells at 1 day and 3 days.

FIG. 9A depicts cell flowmetry data of an embodiment of a hydrogel particle before and after conjugation to RGD for MDA cells.

FIG. 9B depicts cell flowmetry data of an embodiment of a hydrogel particle before and after conjugation to RGD for 3T3 human fibroblasts.

FIG. 10A depicts the distribution at a tumor site upon system administration of saline and embodiments of hydrogel particles, including a redox sensitive hydrogel particle containing RGD (Redox-NPs-RGD), a redox sensitive hydrogel particle containing RGD and doxorubicin (Redox-NPs-RGD-Dox), and a non-redox sensitive hydrogel particle containing RGD and doxorubicin (No Redox-NPs-RGD-Dox).

FIG. 10B depicts the release of free doxorubicin (Hyd-free Dox) and embodiments of hydrogel nanoparticles from a locally implanted composition that includes an embodiment of a host hydrogel in which the embodiments of hydrogel nanoparticles are dispersed. The embodiments of hydrogel nanoparticles (NPs) dispersed in the host hydrogel (Hyd) include a redox sensitive hydrogel particle containing RGD (Hyd-Redox-NPs-RGD), a redox sensitive hydrogel particle containing RGD and doxorubicin (Hyd-Redox-NPs-RGD-Dox), and a non-redox sensitive hydrogel particle containing RGD and doxorubicin (Hyd-No Redox-NPs-RGD-Dox).

FIG. 10C depicts tumor size at days 0-11 after system administration of saline and embodiments of hydrogel particles (NPs), including a redox sensitive hydrogel particle containing RGD (Redox-NPs-RGD), a redox sensitive hydrogel particle containing RGD and doxorubicin (Redox-NPs-RGD-Dox), and a non-redox sensitive hydrogel particle containing RGD and doxorubicin (No Redox-NPs-RGD-Dox).

FIG. 10D depicts tumor size at days 0-11 after the local administration of a composition that includes an embodiment of a host hydrogel in which embodiments of hydrogel nanoparticles are dispersed. The embodiments of hydrogel nanoparticles (NPs) dispersed in the host hydrogel (Hyd) include a redox sensitive hydrogel particle containing RGD (Hyd-Redox-NPs-RGD), a redox sensitive hydrogel particle containing RGD and doxorubicin (Hyd-Redox-NPs-RGD-Dox), and a non-redox sensitive hydrogel particle containing RGD and doxorubicin (Hyd-No Redox-NPs-RGD-Dox).

### DETAILED DESCRIPTION

Improved compositions are provided that may controllably and/or selectively deliver one or more therapeutics to a target site, such as a tumor, due at least in part to the pH and/or redox sensitivity of the compositions. Typically, cancerous tissue presents a more acidic environment than healthy tissue, and the reductant concentration of a tumor microenvironment (both extracellularly and intracellularly) is usually about seven times (7×) greater than that of healthy tissues. Embodiments of the compositions provided herein may exploit at least one of these features of a cancerous tissue in order to controllably, selectively, and/or sustainably delivery one or more drugs to cancer cells. The compositions provided herein also may be biocompatible and/or bioabsorbable.

The compositions provided herein may rely on two distinct release kinetics. One or more drugs may be sustainably and/or locally delivered to the tumor in a selective manner. Following cellular uptake, the hydrogel particles provided herein may have a dual sensitivity that may initiate and/or accelerate drug release to ensure effective disease (e.g., cancer) abrogation.

Generally, in embodiments, the compositions provided herein may be used on or in any diseased cells and/or biological tissue that may include or may be associated with diseased cells. The diseased cells may include cancer cells. For example, the compositions may be used on or in any internal or external biological tissues, lumens, orifices, or cavities. The biological tissues may be those of a human or other mammal, e.g., a patient in need of treatment.

In some embodiments, the compositions include a hydrogel particle, and the hydrogel particle may be dispersed in a host hydrogel. The host hydrogel may serve as a matrix material for controlled delivery of a hydrogel particle. The compositions can be applied to a tissue site in a human or other animal patient, for example, during a surgical or other medical procedure. For example, the compositions may be applied to a tumor or a tissue bed following resection of a tumor.

### Hydrogel Particles

Provided herein are compositions that include hydrogel particles. The hydrogel particles may include (i) a crosslinked polymer that includes a crosslinker, and (ii) a drug conjugated to the polymer component by a covalent bond that is pH sensitive or redox sensitive. The crosslinker may include a moiety that is redox sensitive when the covalent bond is pH sensitive, or the crosslinker may include a moiety that is pH sensitive when the covalent bond is redox sensitive. A cell-penetrating peptide also may be conjugated to the crosslinked polymer.

In one embodiment, the hydrogel particles include (i) a crosslinked polymer that includes a crosslinker having a redox sensitive moiety, and (ii) a drug conjugated to the polymer component by a covalent bond that is pH sensitive. In a further embodiment, the hydrogel particles include (i) a crosslinked polymer that includes a crosslinker having a redox sensitive moiety, (ii) a drug conjugated to the polymer component by a covalent bond that is pH sensitive, and (iii) a cell-penetrating peptide conjugated to the crosslinked polymer.

In one embodiment, the hydrogel particles include (i) a crosslinked polymer that includes a crosslinker having a pH sensitive moiety, and (ii) a drug conjugated to the polymer component by a covalent bond that is redox sensitive. In another embodiment, the hydrogel particles include (i) a crosslinked polymer that includes a crosslinker having a pH sensitive moiety, (ii) a drug conjugated to the polymer component by a covalent bond that is redox sensitive, and (iii) a cell-penetrating peptide conjugated to the crosslinked polymer.

A “covalent bond that is pH sensitive” generally includes a covalent bond, including a reversible covalent bond, that will, or is more likely to, cleave at a particular pH or pH range. For example, a covalent bond that is pH sensitive may include a reversible covalent bond that becomes more likely to cleave at a relatively lower pH, or as the pH of its environment decreases. In one embodiment, the pH-sensitive covalent bond is an imine bond. The likelihood of an imine bond cleaving typically increases as the pH of its environment decreases. Similarly, a “pH sensitive moiety” typically includes two or more covalently bonded atoms that will, or are more likely to, be cleaved upon exposure to a particular pH or pH range. For instance, a pH sensitive moiety may include an imine.

A “covalent bond that is redox sensitive” generally includes a covalent bond that will, or is more likely to, cleave upon contact with a redox agent. In one embodiment, the redox sensitive covalent bond is a disulfide bond. A disulfide bond can be cleaved in the presence of one or more reductants, such as glutathione (GSH), dithiothreitol (DTT), or a combination thereof. Similarly, a “redox sensitive moiety” typically includes two or more covalently bonded atoms that will, or are more likely to, be cleaved upon contacting a redox agent. For instance, a redox sensitive moiety may include a disulfide.

The hydrogel particles generally may be any size capable of being locally delivered to a target site, such as a tumor. The size of the hydrogel particles may be limited only by the ability of the hydrogel particles to be uptaken by one or more diseased cells. When the compositions provided herein include a plurality of hydrogel particles, the compositions may include hydrogel particles of two or more sizes. In embodiments, the hydrogel particles have an average diameter of about 50 nm to about 1,500 nm. In some embodiments, the hydrogel particles have an average diameter of about 100 nm to about 1,300 nm. In one embodiment, the hydrogel particles have an average diameter of about 115 nm to about 155 nm, about 125 nm to about 145 nm, or about 135 nm. In another embodiment, the hydrogel particles have an average diameter of about 140 nm to about 160 nm, about 145 nm to about 155 nm, or about 150 nm. In a further embodiment, the hydrogel particles have an average diameter of about 160 nm to about 200 nm, about 170 nm to about 190 nm, or about 180 nm. In a particular embodiment, the hydrogel particles have an average diameter of about 575 nm to about 675 nm, about 600 nm to about 650 nm, or about 625 nm. In an additional embodiment, the hydrogel particles have an average diameter of about 925 nm to about 1,050 nm, about 950 nm to about 1,025 nm, about 975 nm to about 1,005 nm, or about 990 nm. In a still further embodiment, the hydrogel particles have an average diameter of about 900 nm to about 1,300 nm, about 1,000 nm to about 1,200 nm, about 1,050 nm to about 1,150 nm, or about 1,100 nm. The average size of the hydrogel particles may be determined by dynamic light scattering, as explained herein at Example 1. Not wishing to be bound by any particular theory, the average size of the hydrogel particles may be controlled by adjusting the polymer/crosslinker ratio and/or the water/oil ratio when the hydrogel particles are made with a single-emulsion technique, as described herein.

The hydrogel particles generally may be of any shape. The hydrogel particles may include a single shape or two or more shapes. In one embodiment, the hydrogel particles are substantially spherical. In another embodiment, the hydrogel particles are substantially oval-shaped. In yet another embodiment, the hydrogel particles include substantially spherical particles and substantially oval-shaped particles. The hydrogel particles also may have an irregular shape. When the hydrogel particles are or include non-spherical shapes, the phrase “average diameter” refers to the average largest dimension of the hydrogel particles.

The crosslinked polymer of the hydrogel particles generally may include any one or more polymers capable of forming a hydrogel. In embodiments, the crosslinked polymer includes one or more functional groups that permit the crosslinked polymer to react with and form covalent bonds with a crosslinker and a drug that is conjugated to the crosslinked polymer. The one or more functional groups of the crosslinked polymer may include one type of functional group or two or more types of functional groups. For example, the crosslinked polymer may include one type of functional group that is capable of reacting with and forming a covalent bond with both the crosslinker and the drug; or, the crosslinked polymer may include one type of functional group that is capable of reacting with and forming a covalent bond with a crosslinker, and a second type of functional group that is capable of reacting with and forming covalent bond with a drug. Therefore, the type of functional groups may be used, at least in part, to control the degree of crosslinking, the amount of drug and/or cell-penetrating peptide conjugated to the crosslinked polymer, or a combination thereof. A drug may be modified to include a functional group that is capable of reacting with and forming a covalent bond with a functional group of the crosslinked polymer. Generally, a drug may be modified in any manner that does not substantially impact its therapeutic efficacy. For example, a drug may be modified with one or more small molecules, such as amino acids, that include a functional group, such as an amine, that may be capable of reacting with and forming a covalent bond with a functional group of the crosslinked polymer.

In embodiments, the crosslinked polymer includes one or more aldehydes. The one or more aldehydes may react with amine groups to form imine bonds. When the crosslinked polymer includes one or more aldehydes, the crosslinker may include at least two terminal amines, and the drug may include, or be modified to include, an amine. For example, the drug may include doxorubicin, which includes a primary amine that is capable of reacting with an aldehyde to form a pH sensitive covalent bond, i.e., an imine.

The crosslinked polymer generally may be selected from at least one polysaccharide, at least one hydrophilic polymer, at least one hydrophobic polymer, or combinations thereof.

In one embodiment, the crosslinked polymer is a polysaccharide having one or more aldehyde groups. In a certain embodiment, the crosslinked polymer is a hydrophilic polymer having one or more aldehyde groups. In a particular embodiment, the crosslinked polymer includes a polysaccharide having one or more aldehyde groups, and a hydrophilic polymer having one or more aldehyde groups. The polysaccharide may be linear, branched, or have both linear and branched sections within its structure. The polysaccharide may be anionic, cationic, nonionic, or a combination thereof. Generally, the polysaccharide may be natural, synthetic, or modified—for example, by altering the polysaccharide's substituents, or a combination thereof. In one embodiment, the polysaccharide is plant-based. In another embodiment, the polysaccharide is animal-based. In yet another embodiment, the polysaccharide is a combination of plant-based and animal-based polysaccharides. Non-limiting examples of polysaccharides include, but are not limited to, dextran, dextrin, chitin, starch, agar, cellulose, hyaluronic acid, derivatives thereof, such as cellulose derivatives, or a combination thereof.

In embodiments, the polysaccharide is nonionic. Non-limiting examples of nonionic polysaccharides include dextran, dextrin, and cellulose derivatives. In other embodiments, the polysaccharide is anionic. Non-limiting examples of anionic polysaccharides include hyaluronic acid, chondroitin sulfate, alginate, and cellulose gum. In further embodiments, the polysaccharide is cationic. The cationic character may be imparted by substituting the polysaccharide with positively charge groups, such as trimethylammonium groups. Non-limiting examples of cationic polysaccharides include chitosan, cationic guar gum, cationic hydroxyethylcellulose, or other polysaccharides modified with trimethylammonium groups to confer positive charge.

In embodiments, the crosslinked polymer includes one or more hydrophilic polymers. The hydrophilic polymers are modified, in some embodiments, to confer degradability. For example, the hydrophilic polymers may be modified with polyester groups in order to impart degradability of the hydrophilic polymer. Generally, any biocompatible hydrophilic polymer may be used. Non-limiting examples of hydrophilic polymers include poly(vinyl alcohol), poly(acrylic acid), poly(acrylamide), poly(ethylene oxide), or combinations thereof.

In embodiments, the crosslinked polymer includes one or more hydrophobic polymers. The hydrophobic polymers may be modified with pendant hydrophilic polymers to adjust their characteristics. Non-limiting examples of hydrophobic polymers include polycaprolactam, poly(lactic acid), polycaprolactone, or combinations thereof.

In embodiments, the polymer of the crosslinked polymer has a molecular weight of about 1,000 to about 1,000,000 Daltons, or about 5,000 to about 15,000 Daltons, prior to crosslinking and conjugation of a drug and/or cell-penetrating peptide. Unless specified otherwise, the “molecular weight” of the polymer refers to the number average molecular weight. The molecular weight may be adjusted to attain certain properties, as known to those of skill in the art.

Generally, the one or more functional groups of the crosslinked polymer may be present in a number sufficient to form the hydrogel particles described herein. In certain embodiments, the crosslinked polymer's degree of functionalization is adjustable. The “degree of functionalization” generally refers to the number or percentage of groups on the crosslinked polymer that are replaced or converted to the desired one or more functional groups. The one or more functional groups, in particular embodiments, include aldehydes. In one embodiment, the degree of functionalization is adjusted based on the desired degree of crosslinking, the desired amount of drug and/or cell-penetrating peptide to be conjugated to the crosslinked polymer, or a combination thereof. In one embodiment, the degree of functionalization is about 10% to about 75%. In another embodiment, the degree of functionalization is about 25% to about 60%. In yet another embodiment, the degree of functionalization is about 40% to about 50%. In a still further embodiment, the degree of functionalization is about 50%.

In one embodiment, the crosslinked polymer is a polysaccharide having about 10% to about 75% of its vicinal hydroxyl groups converted to aldehydes. In another embodiment, the crosslinked polymer is a polysaccharide having about 25% to about 75% of its vicinal hydroxyl groups converted to aldehydes.

In one embodiment, the polymer of the crosslinked polymer is dextran with a molecular weight of about 10 kDa prior to crosslinking and conjugation of a drug and/or cell-penetrating peptide. In another embodiment, the polymer of the crosslinked polymer is dextran having about 50% of its vicinal hydroxyl group converted to aldehydes. In a further embodiment, the polymer of the crosslinked polymer is dextran with a molecular weight of about 10 kDa (prior to crosslinking and conjugation of a drug and/or cell-penetrating peptide) and about 50% of its vicinal hydroxyl groups converted to aldehydes.

In some embodiments, a polysaccharide and/or hydrophilic polymer is oxidized to include a desired percentage of one or more aldehyde functional groups. Generally, this oxidation may be conducted using any known means. For example, suitable oxidizing agents include, but are not limited to, periodates, hypochlorites, ozone, peroxides, hydroperoxides, persulfates, and percarbonates. In one embodiment, the oxidation is performed using sodium periodate. Typically, different amounts of oxidizing agents may be used to alter the degree of functionalization. In addition to, or independently of, other methods, aldehyde groups can be grafted onto the polymer backbone using known bioconjugation techniques in the event that oxidative methods are unsuitable.

Generally, any drug may be conjugated to the crosslinked polymer. The drug may include a single type or drug or two or more types of drug. The amount of drug conjugated to the crosslinked polymer may be limited only by the number of available functional groups of the crosslinked polymer. The amount of drug conjugated to the crosslinked polymer may be selected based on therapeutic efficacy, a desired release profile, or a combination thereof. In embodiments, the molar ratio of drug to polymer of the crosslinked polymer (for example, dextran prior to crosslinking and possible cell-penetrating peptide conjugation) is about 0.01:1 to about 1:1, about 0.1:1 to about 1:1, about 0.3:1 to about 1:1, about 0.5:1 to about 1:1, or about 0.8:1 to about 1:1.

A first molecule, such as a drug and/or cell-penetrating peptide, is “conjugated” to a crosslinked polymer, when an atom of the first molecule is covalently bonded to an atom of the crosslinked polymer. The first molecule may be modified to include a functional group that is capable of reacting with and covalently bonding to the crosslinked polymer. For example, the drug and/or cell-penetrating peptide may be modified to include a linker molecule. The linker molecule may include one or more amino acids. Therefore, the pH sensitive or redox sensitive covalent bond that connects a drug to a crosslinked polymer may be a covalent bond between [1] an atom of a drug and an atom of a crosslinked polymer, [2] an atom of a linker and an atom of a drug, [3] an atom of a linker and an atom of a crosslinked polymer, [4] two atoms of a linker, or [5] a combination thereof.

In embodiments, a cell-penetrating peptide is conjugated to the crosslinked polymer. The cell-penetrating peptide may be conjugated to the crosslinked polymer via the same functional groups that are capable of covalently bonding with a drug, a crosslinker, or a combination thereof. A “cell-penetrating peptide” generally is any molecule having the ability to increase the cellular uptake of a hydrogel particle. In one embodiment, the cell-penetrating peptide increases the likelihood that the hydrogel particles are selectively uptaken by a diseased cell, such as a cancer cell. The cell-penetrating peptide may be conjugated to the hydrogel particles at any point during the production process. In a particular embodiment, the cell-penetrating peptide is conjugated to the hydrogel particles after particle formation, and the concentration of conjugated cell-penetrating peptides is the greatest on the surfaces of the hydrogel particles. In one embodiment, the cell-penetrating peptide comprises RGD (arginylglycylaspartic acid). Not wishing to be bound by any particular theory, RGD is recognized by cell-surface receptors, such as integrins, which are known to mediate cell adhesion and proliferation. These integrins typically are expressed specifically on proliferating endothelial cells, such as those present in tumors, and, therefore, represent a marker for malignancy.

One or more of the hydrogel particles described herein may be administered systemically, locally, or a combination thereof to a patient, including a human. One or more of the hydrogel particles may be dispersed in a medium, such as a host hydrogel, prior to being locally applied to one or more biological tissues. Upon or after being applied to one or more biological tissues, the hydrogel particles may be permitted to diffuse from the medium, such as a host hydrogel, and into the biological tissue. A hydrogel particle diffuses “into a biological tissue” when the hydrogel particle contacts a biological tissue, penetrates a biological tissue, such as a cell wall, or a combination thereof. The biological tissue, which may include any mammalian tissue, may include or be associated with one or more diseased cells, such as cancer cells.

When one or more hydrogel particles diffuses into a biological tissue, the environment in or adjacent to one or more diseased cells, such as cancer cells, may trigger at least one of the pH sensitive and redox sensitive features of the hydrogel particles, thereby causing and/or accelerating drug release. When the one or more diseased cells includes a tumor cell, the tumor cell may have a reduced pH and/or reducing environment that triggers drug release from the one or more hydrogel particles. Therefore, the one or more hydrogel particles may be dispersed in a medium, and administered to a patient in an effective amount to treat cancer. The one or more hydrogel particles dispersed in a medium, such as a host hydrogel, may be administered locally to one or more tumors in the patient's body.

### Crosslinkers

The crosslinkers of the crosslinked polymers of the hydrogel particles provided herein may include [1] two or more moieties capable of forming a covalent bond with a polymer of the hydrogel particles, and [2] a redox sensitive moiety or a pH sensitive moiety. The two or more moieties capable of forming a covalent bond with a polymer of the hydrogel particles may be the same or different. The amount of crosslinker that may be used to crosslink a crosslinked polymer may be limited only by the number and/or availability of functional groups of a polymer. In embodiments, the molar ratio of polymer to crosslinker is about 1:1 to about 1:10, about 1:1 to about 1:5, or about 1:5 to about 1:10.

In embodiments, the redox sensitive moiety includes one or more disulfide bonds. Therefore, each molecule of a crosslinker may include one, two, three, or more disulfide bonds.

In embodiments, the redox sensitive moiety includes one or more disulfide bonds, and the two or more moieties capable of forming a covalent bond with a polymer comprise amines, such as primary amines. In one embodiment, the crosslinker comprises a compound having the following structure:

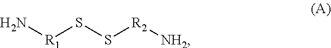

wherein R₁ and R₂ are independently selected from a divalent C₁-C₂₀ hydrocarbyl. The primary amines of formula (A) may react with and form covalent bonds with a polymer of a hydrogel particle.

In one embodiment, the crosslinker comprises a compound according to formula (A), wherein R₁ and R₂ are unsubstituted divalent C₁-C₅ hydrocarbyls, and the crosslinker has the following structure:

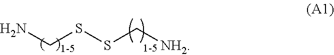

In one embodiment, the crosslinker comprises a compound of formula (A1), wherein R₁ and R₂ are unsubstituted divalent C₂ hydrocarbyls, and the crosslinker comprises cystamine (i.e., 2,2′-disulfanediyldiethanamine):

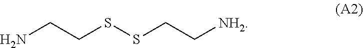

Although the crosslinkers herein may be shown in their independent, unreacted form, it is understood by persons of ordinary skill in the art that the chemical structures of the crosslinkers typically are altered when one or both of their ends groups react with a polymer of a hydrogel particle to form a crosslinked polymer. For example, when [1] a crosslinker comprises formula (A2), and [2] a polymer includes aldehydes, then the amines of the crosslinker may react with aldehydes of the polymer to form the following structure:

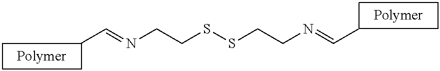

### Drugs

Generally, any drug may be conjugated to a crosslinked polymer of a hydrogel particle. The drug that is conjugated to a crosslinked polymer of a hydrogel particle may include a single type of drug or two or more types of drug. Since the response to drugs can vary from patient to patient, the compositions provided herein may be personalized on a patient-by-patient basis. In embodiments, the drug includes one or more anti-tumor agents, one or more anti-angiogenic agents, or a combination thereof. The drug may be modified to permit its conjugation to a crosslinked polymer.

The one or more anti-tumor agents may include one or more chemotherapeutic agents, one or more peptides, one or more proteins, one or more antibodies, one or more nucleic acids, or a combination thereof.

In embodiments, the drug conjugated to a crosslinked polymer of a hydrogel particle includes one or more chemotherapeutic agents. A “chemotherapeutic agent” is a chemical compound useful in the treatment of cancer. Examples of chemotherapeutic agents include, but are not limited to, alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrirnidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In one embodiment, the one or more chemotherapeutic agents includes an anthracycline drug. In a particular embodiment, the one or more chemotherapeutic agents includes doxorubicin.

In embodiments, the drug includes an anti-angiogenic agent. An “anti-angiogenic agent” includes drugs that inhibit the growth of blood vessels. In one embodiment, the anti-angiogenic agent is bevacizumab (Avastin®)). Other anti-angiogenic agents that may be conjugated to the first metal nanoparticles provided herein include, but are not limited to, axitinib, cabozantinib, cetuximab, everolimus, lenalidomide, pazopanib, ramucirumab, regorafenib, sorafenib, sunitinib, thalidomide, vandetanib, and ziv-aflibercept.

### Host Hydrogel

One or more of the hydrogel particles provided herein may be dispersed in a host hydrogel. The host hydrogel may permit the hydrogel particles to be locally released to a target site, such as a tumor tissue, in a sustained manner due to the sustained release of the hydrogel particles from the hydrogel scaffold. The release of hydrogel particles from a host hydrogel following deployment may be continuous, intermittent, and/or delayed for a desired time after deployment. The hydrogel particles released from a host hydrogel can be uptaken, possibly selectively, by one or more diseased cells, such as a cancer cell, and the hydrogel particles, as described herein, may release drug and/or the release of drug may be accelerated as the pH sensitive and/or redox sensitive features of the hydrogel particles are triggered by one or more stimuli.

In embodiments, the compositions provided herein include a host hydrogel. One or more hydrogel particles may be dispersed in the host hydrogel. The one or more hydrogel particles may be dispersed at least substantially uniformly in the host hydrogel, or non-uniformly in the host hydrogel. The concentration of the hydrogel particles in the host hydrogel may be about 1 mg/mL to about 75 mg/mL, about 1 mg/mL to about 50 mg/mL, about 5 mg/mL to about 50 mg/mL, about 10 mg/mL to about 50 mg/mL, about 20 mg/mL to about 50 mg/mL, or about 25 mg/mL to about 50 mg/mL.

Generally, the compositions described herein may include any biocompatible host hydrogel. In such embodiments, the host hydrogel may serve as a matrix material for controlled delivery of drug, localized drug delivery, or a combination thereof. Methods of locally delivering a drug may include applying to a biological tissue in vivo, such as a human tissue, a drug delivery composition as provided herein, and permitting the hydrogel particles to which a drug is conjugated to diffuse from the composition into the biological tissue. The host hydrogel may adhere to one or more biological tissues, thereby reducing or eliminating the risk of unwanted material migration following application of the composition to one or more selected tissue sites. The host hydrogel generally may be degradable, injectable, or a combination thereof.

The host hydrogel may include a contact product of [1] a first solution that includes the first polymer component described herein, and [2] a second solution that includes the second polymer component and/or the dendrimer component described herein. One or more hydrogel particles may be added to the host hydrogel after hydrogel formation; the one or more hydrogel particles may be added to the first solution, the second solution, or both the first and second solution prior to host hydrogel formation; or a combination thereof. The one or more hydrogel particles may be present in the first solution, the second solution, or a combination thereof in an amount sufficient to impart the resulting host hydrogel with a concentration of the hydrogel particles of about 1 mg/mL to about 75 mg/mL, about 1 mg/mL to about 50 mg/mL, about 5 mg/mL to about 50 mg/mL, about 10 mg/mL to about 50 mg/mL, about 20 mg/mL to about 50 mg/mL, or about 25 mg/mL to about 50 mg/mL. The hydrogel particles may be disposed in the solution having components with which the hydrogel particles are incapable of reacting. For example, if [1] the second solution includes a component that includes amines, and [2] the hydrogel particles include aldehydes and at least a portion of those aldehydes are not covalently bonded to a drug, crosslinker, and/or cell-penetrating peptide, then the hydrogel particles may be disposed in the first solution to avoid the possibility of the hydrogel particles reacting with the amines of the second solution prior to host hydrogel formation. If, however, a reaction between the hydrogel particles and a component of the host hydrogel may be beneficial prior to host hydrogel formation, then the hydrogel particles may be disposed in the second solution.

The rate of drug delivery may be controlled, at least in part, by imparting the hydrogel particles with one or more functional groups capable of reacting with a functional group of at least one component of the host hydrogel in which the hydrogel particles are dispersed. If the hydrogel particles do not include functional groups capable of reacting with a functional group of at least one component of the host hydrogel in which the hydrogel particles are dispersed, then the rate of drug delivery may be dictated by the diffusion of the one or more hydrogel particles from the host hydrogel and the triggering of the redox sensitive and/or pH sensitive features of the hydrogel particles. If the hydrogel particles do include a functional group capable of reacting with a functional group of at least one component of the host hydrogel, then the rate of drug delivery may be dictated by the degradation rate of the host hydrogel, the diffusion of the hydrogel particles from the host hydrogel, the triggering of the redox sensitive and/or pH sensitive feature of the hydrogel particles, or a combination thereof.

Generally, the hydrogel composites and compositions, including drug delivery compositions, provided herein may be formed by combining a first solution and a second solution as described herein. The first solution and the second solution may be aqueous macromer solutions. The first solution and/or the second solution may independently include water, phosphate buffer saline (PBS), Dulbecco's Modified Eagle's Medium (DMEM), or any combination thereof.

The first solution, in embodiments, includes one or more hydrogel particles and a first polymer component. The first solution, in other embodiments, includes a first polymer component without one or more hydrogel particles.

The second solution may include at least one of a dendrimer and a second polymer component. The dendrimer and/or second polymer component generally have one or more functional groups capable of reacting with the one or more functional groups on the first polymer. The dendrimer and/or second polymer component, in particular embodiments, include one or more amines. The second solution, in other embodiments, also includes the one or more hydrogel particles.

The first solution and the second solution, in embodiments, are combined to form the hydrogel composites and compositions described herein. When combined, the aldehyde groups of the first solution may react with the amines that are present in the second solution. This reaction is referred to herein as “curing” or “gelling.”

In embodiments, one or more hydrogel particles are present in the first solution. In some embodiments, one or more hydrogel particles are present in the first solution and the second solution. In further embodiments, one or more hydrogel particles are present in the second solution. When the first solution and the second solution include one or more hydrogel particles, the one or more hydrogel particles of the first solution and the second solution may have the same or different compositions. For example, the first solution may include hydrogel particles conjugated to a first drug, and the second solution may include one or more hydrogel particles conjugated to a second drug. As a further example, the first solution may include one or more hydrogel particles lacking functional groups capable of reacting with the components of the first solution, and the second solution may include one or more hydrogel particles lacking functional groups capable of reacting with the components of the second solution.

In embodiments, the one or more hydrogel particles are substantially evenly dispersed in the first solution. In other embodiments, the one or more hydrogel particles are substantially evenly dispersed in the first solution and the second solution. In further embodiments, the one or more hydrogel particles are evenly dispersed in the second solution. Although the one or more hydrogel particles are evenly dispersed in preferred embodiments, other embodiments may not have an even dispersement of the hydrogel particles.

In embodiments, the concentration of the one or more hydrogel particles in the first solution is about 0.01% to about 30% by weight of the first solution. In some embodiments, the concentration of the one or more hydrogel particles in the first solution is about 0.01% to about 25% by weight of the first solution. In further embodiments, the concentration of the one or more hydrogel particles in the first solution is about 0.01% to about 20% by weight of the first solution. In still further embodiments, the concentration of the one or more hydrogel particles in the first solution is about 0.01% to about 15% by weight of the first solution.

In embodiments, the concentration of the one or more hydrogel particles in the second solution is about 0.01% to about 30% by weight of the second solution. In some embodiments, the concentration of the one or more hydrogel particles in the second solution is about 0.01% to about 25% by weight of the second solution. In further embodiments, the concentration of the one or more hydrogel particles in the second solution is about 0.01% to about 20% by weight of the second solution. In still further embodiments, the concentration of the one or more hydrogel particles in the second solution is about 0.01% to about 15% by weight of the second solution.

In embodiments, the concentration of the one or more hydrogel particles in the hydrogel composites or compositions described herein is about 0.01% to about 10% by weight of the hydrogel composite or composition. In some embodiments, the concentration of the one or more hydrogel particles in the hydrogel composites or compositions described herein is about 0.01% to about 8% by weight of the hydrogel composite or composition. In certain embodiments, the concentration of the one or more hydrogel particles in the hydrogel composites or compositions described herein is about 0.01% to about 6% by weight of the hydrogel composite or composition. In particular embodiments, the concentration of the one or more hydrogel particles in the hydrogel composites or compositions described herein is about 0.01% to about 5% by weight of the hydrogel composite or composition.

In embodiments, the concentration of first polymer component in the first solution is about 0.01% to about 40% by weight of the first solution. In further embodiments, the concentration of first polymer component in the first solution is about 0.01% to about 30% by weight of the first solution. In some embodiments, the concentration of first polymer component in the first solution is about 0.01% to about 20% by weight of the first solution. In a particular embodiment, the concentration of first polymer component in the first solution is about 20% by weight of the first solution. In additional embodiments, the concentration of first polymer component in the first solution is about 0.01% to about 10% by weight of the first solution. Typically, the concentration may be tailored and/or adjusted based on the particular application, tissue type, and/or the type and concentration of dendrimer and/or second polymer component used.

In embodiments, the concentration of the first polymer component in the hydrogel composites or compositions described herein is about 0.01% to about 20% by weight of the hydrogel composite or composition. In further embodiments, the concentration of the first polymer component in the hydrogel composites or compositions described herein is about 0.01% to about 15% by weight of the hydrogel composite or composition. In some embodiments, the concentration of the first polymer component in the hydrogel composites or compositions described herein is about 0.01% to about 10% by weight of the hydrogel composite or composition. In still further embodiments, the concentration of the first polymer component in the hydrogel composites or compositions described herein is about 0.01% to about 7% by weight of the hydrogel composite or composition.

In embodiments, the total concentration of dendrimer and second polymer component in the second solution is about 0.01% to about 40% by weight of the second solution. In further embodiments, the total concentration of dendrimer and second polymer component in the second solution is about 0.01% to about 30% by weight of the second solution. In some embodiments, the total concentration of dendrimer and second polymer component in the second solution is about 0.01% to about 20% by weight of the second solution. In additional embodiments, the total concentration of dendrimer and second polymer component in the second solution is about 0.01% to about 10% by weight of the second solution. In a particular embodiment, the total concentration of dendrimer and second polymer component in the second solution is about 25% by weight of the second solution. Typically, the concentration may be tailored and/or adjusted based on the particular application, tissue type, and/or the type and concentration of first polymer component used. As used herein, the phrase “total concentration of dendrimer and second polymer component” refers to the sum of the concentration of dendrimer and the concentration of the second polymer component. The phrase does not imply that both a dendrimer and a second polymer component must be present in the second solution. The second solution may include a dendrimer, second polymer component, or both a dendrimer and second polymer component.

In embodiments, the total concentration of dendrimer and second polymer component in the hydrogel composites or compositions described herein is about 0.01% to about 20% by weight of the hydrogel composite or composition. In further embodiments, the total concentration of dendrimer and second polymer component in the hydrogel composites or compositions described herein is about 0.01% to about 15% by weight of the hydrogel composite or composition. In some embodiments, the total concentration of dendrimer and second polymer component in the hydrogel composites or compositions described herein is about 0.01% to about 10% by weight of the hydrogel composite or composition. In still further embodiments, the total concentration of dendrimer and second polymer component in the hydrogel composites or compositions described herein is about 0.01% to about 7% by weight of the hydrogel composite or composition.

### First Polymer Component

The first polymer component generally includes a first polymer with one or more functional groups capable of reacting with one or more functional groups on a biological tissue and/or one or more functional groups on the dendrimer component and/or second polymer component of the second solution. The one or more functional groups of the first polymer component also may be capable of reacting with one or more functional groups on the hydrogel particles. The first polymer component, in embodiments, comprises a first polymer having one or more aldehyde groups.

The polymers of the first polymer component may be selected from any biocompatible polymers capable of forming or imparting certain characteristics to the hydrogel composites and compositions described herein. The polymers of the first polymer component, for example, may be selected from at least one polysaccharide, at least one hydrophilic polymer, at least one hydrophobic polymer, or combinations thereof.

In one embodiment, the first polymer component includes a first polymer that is a polysaccharide having one or more aldehyde groups. In a certain embodiment, the first polymer component includes a first polymer that is a hydrophilic polymer having one or more aldehyde groups. In another embodiment, the first polymer component includes a first polymer that is a polysaccharide having one or more aldehyde groups, and a hydrophilic polymer. In further embodiments, the first polymer component includes a first polymer that is a polysaccharide having one or more aldehyde groups, a hydrophilic polymer, and a hydrophobic polymer. In some embodiments, the first polymer component comprises a first polymer that includes a polysaccharide and a hydrophilic polymer, wherein both the polysaccharide and hydrophilic polymer have one or more aldehyde groups. Therefore, as used herein, the phrase “first polymer” refers to the one or more polymers of the first polymer component that include one or more functional groups, e.g., aldehydes, that are capable of reacting with a biological tissue and/or the functional groups of the dendrimer component and/or second polymer component. In still further embodiments, the first polymer component comprises a first polymer that includes a polysaccharide and a hydrophilic polymer, wherein both the polysaccharide and hydrophilic polymer have one or more aldehyde groups, and a hydrophobic polymer.

In embodiments, the first polymer comprises at least one polysaccharide. The at least one polysaccharide may be linear, branched, or have both linear and branched sections within its structure. The at least one polysaccharide may be anionic, cationic, nonionic, or a combination thereof. Generally, the at least one polysaccharide may be natural, synthetic, or modified—for example, by crosslinking, altering the polysaccharide's substituents, or both. In one embodiment, the at least one polysaccharide is plant-based. In another embodiment, the at least one polysaccharide is animal-based. In yet another embodiment, the at least one polysaccharide is a combination of plant-based and animal-based polysaccharides. Non-limiting examples of polysaccharides include, but are not limited to, dextran, dextrin, chitin, starch, agar, cellulose, hyaluronic acid, derivatives thereof, such as cellulose derivatives, or a combination thereof.

In embodiments, the at least one polysaccharide is nonionic. Non-limiting examples of nonionic polysaccharides include dextran, dextrin, and cellulose derivatives. In other embodiments, the at least one polysaccharide is anionic. Non-limiting examples of anionic polysaccharides include hyaluronic acid, chondroitin sulfate, alginate, and cellulose gum. In further embodiments, the at least one polysaccharide is cationic. The cationic character may be imparted by substituting the at least one polysaccharide with positively charge groups, such as trimethylammonium groups. Non-limiting examples of cationic polysaccharides include chitosan, cationic guar gum, cationic hydroxyethylcellulose, or other polysaccharides modified with trimethylammonium groups to confer positive charge.

In embodiments, the first polymer component comprises one or more hydrophilic polymers. The hydrophilic polymers are modified, in some embodiments, to confer degradability. For example, the hydrophilic polymers may be modified with polyester groups in order to impart degradability of the hydrophilic polymer. In particular embodiments, the hydrophilic polymers are substituted with one or more functional groups, such as aldehydes, that are capable of reacting with biological tissue and/or the functional groups of the dendrimer and/or second polymer component, such as amines. Generally, any biocompatible hydrophilic polymer may be used. Non-limiting examples of hydrophilic polymers include poly(vinyl alcohol), poly(acrylic acid), poly(acrylamide), poly(ethylene oxide), or combinations thereof.

In embodiments, the first polymer component comprises one or more hydrophobic polymers. The hydrophobic polymers may be modified with pendant hydrophilic polymers to adjust their characteristics. Non-limiting examples of hydrophobic polymers include polycaprolactam, poly(lactic acid), polycaprolactone, or combinations thereof.

In certain embodiments, the first polymer has a molecular weight of about 1,000 to about 1,000,000 Daltons. In one embodiment, the first polymer has a molecular weight of about 5,000 to about 15,000 Daltons. Unless specified otherwise, the “molecular weight” of the polymer refers to the number average molecular weight. The molecular weight may be adjusted to attain certain properties, as known to those of skill in the art.

Generally, the one or more functional groups of the first polymer may be present in a number sufficient to form the hydrogel composites and compositions described herein. In certain embodiments, the first polymer's degree of functionalization is adjustable. The “degree of functionalization” generally refers to the number or percentage of groups on the polymer that are replaced or converted to the desired one or more functional groups. The one or more functional groups, in particular embodiments, include aldehydes. In one embodiment, the degree of functionalization is adjusted based on the type of tissue to which the hydrogel composites or compositions is applied, the concentration(s) of the various components, and/or the type of polymer(s) or dendrimer(s) used in the first and second solutions. In one embodiment, the degree of functionalization is about 10% to about 75%. In another embodiment, the degree of functionalization is about 25% to about 60%. In yet another embodiment, the degree of functionalization is about 40% to about 50%.

In one embodiment, the first polymer is a polysaccharide having about 10% to about 75% of its vicinal hydroxyl groups converted to aldehydes. In another embodiment, the first polymer is a polysaccharide having about 25% to about 75% of its vicinal hydroxyl groups converted to aldehydes.

In one embodiment, the first polymer is dextran with a molecular weight of about 10 kDa. In another embodiment, the first polymer is dextran having about 50% of its vicinal hydroxyl group converted to aldehydes. In a further embodiment, the first polymer is dextran with a molecular weight of about 10 kDa and about 50% of its vicinal hydroxyl groups converted to aldehydes.

In some embodiments, a polysaccharide and/or hydrophilic polymer is oxidized to include a desired percentage of one or more aldehyde functional groups. Generally, this oxidation may be conducted using any known means. For example, suitable oxidizing agents include, but are not limited to, periodates, hypochlorites, ozone, peroxides, hydroperoxides, persulfates, and percarbonates. In one embodiment, the oxidation is performed using sodium periodate. Typically, different amounts of oxidizing agents may be used to alter the degree of functionalization. In addition to, or independently of, other methods, aldehyde groups can be grafted onto the polymer backbone using known bioconjugation techniques in the event that oxidative methods are unsuitable.

### Second Polymer Component

The second polymer component generally includes a second polymer with one or more functional groups capable of reacting with one or more functional groups of the first polymer of the first polymer component. The second polymer component, in embodiments, comprises a second polymer having one or more amines. The amines may be primary amines, secondary amines, or a combination thereof.

The polymers of the second polymer component may be selected from any biocompatible polymers capable of forming or imparting certain characteristics to the hydrogel composites and compositions described herein. The polymers of the second polymer component, for example, may be selected from at least one biopolymer, polyamine, or a combination thereof.

In one embodiment, the second polymer component includes a second polymer that is a biopolymer having one or more amines, such as primary amines, secondary amines, or a combination thereof. Non-limiting examples of biopolymers include chitosan, collagen, gelatin, other structural biomolecules, or a combination thereof. In a particular embodiment, the second polymer comprises a polyamine. The polyamine may be synthetic. Non-limiting examples of polyamines include amine-terminated, multi-arm poly(ethylene oxide) and polyethyleneimine. In another embodiment, the second polymer component includes a second polymer that comprises both (i) a biopolymer having one or more amines, and (ii) a polyamine. Therefore, as used herein, the phrase “second polymer” refers to the one or more polymers of the second polymer component that include one or more functional groups, e.g., amines, that are capable of reacting with the one or more functional groups of the first polymer component, such as aldehydes.

In some embodiments, the second polymer is a commercially available amine-terminated polymer, such as Type I collagen, Type II collagen, Type III collagen, gelatin that is acid- or base-catalyzed (i.e., Type A or Type B), or 10 kD dextran (Pharmacosmos AIS, Denmark).

### Dendrimer Component

In embodiments, the second solution comprises a dendrimer component. The dendrimer component of the second solution may include a dendrimer as described herein. The dendrimer may be substituted with one or more functional groups, such as amines, that are capable of reacting with the one or more functional groups of the first polymer of the first polymer component.

In some embodiments, the dendrimer has amines on at least a portion of its surface groups, which are commonly referred to as “terminal groups” or “end groups.” The dendrimer may have amines on from 25% to 100% of its surface groups. In some embodiments, the dendrimer has amines on 100% of its surface groups. In one embodiment, the dendrimer has amines on less than 75% of its surface groups. As used herein, the term “dendrimer” refers to any compound with a polyvalent core covalently bonded to two or more dendritic branches. In some embodiments, the polyvalent core is covalently bonded to three or more dendritic branches. In one embodiment, the amines are primary amines. In another embodiment, the amines are secondary amines. In yet another embodiment, one or more surface groups have at least one primary and at least one secondary amine.

In one embodiment, the dendrimer extends through at least 2 generations. In another embodiment, the dendrimer extends through at least 3 generations. In yet another embodiment, the dendrimer extends through at least 4 generations. In still another embodiment, the dendrimer extends through at least 5 generations. In a further embodiment, the dendrimer extends through at least 6 generations. In still a further embodiment, the dendrimer extends through at least 7 generations.

In one embodiment, the dendrimer has a molecular weight of about 1,000 to about 1,000,000 Daltons. In a further embodiment, the dendrimer has a molecular weight of about 3,000 to about 120,000 Daltons. In another embodiment, the dendrimer has a molecular weight of about 10,000 to about 100,000 Daltons. In yet another embodiment, the dendrimer has a molecular weight of about 20,000 to about 40,000 Daltons. Unless specified otherwise, the “molecular weight” of the dendrimer refers to the number average molecular weight.

Generally, the dendrimer may be made using any known methods. In one embodiment, the dendrimer is made by oxidizing a starting dendrimer having surface groups comprising at least one hydroxyl group so that at least a portion of the surface groups comprise at least one amine. In another embodiment, the dendrimer is made by oxidizing a starting generation 5 (G5) dendrimer having surface groups comprising at least one hydroxyl group so that at least a portion of the surface groups comprise at least one amine. In yet another embodiment, the dendrimer is made by oxidizing a starting G5 dendrimer having surface groups comprising at least one hydroxyl group so that about 25% to 100% of the surface groups comprise at least one amine. In a particular embodiment, the dendrimer is a G5 dendrimer having primary amines on about 25% to 100% of the dendrimer's surface groups. In a certain embodiment, the dendrimer is a G5 dendrimer having primary amines on about 25% of the dendrimer's surface groups.

In one embodiment, the dendrimer is a poly(amidoamine)-derived (PAMAM) dendrimer. In another embodiment, the dendrimer is a G5 PAMAM-derived dendrimer. In yet another embodiment, the dendrimer is a G5 PAMAM-derived dendrimer having primary amines on about 25% to 100% of the dendrimer's surface groups. In a further embodiment, the dendrimer is a G5 PAMAM-derived dendrimer having primary amines on about 25% of the dendrimer's surface groups.

In one embodiment, the dendrimer is a poly(propyleneimine)-derived dendrimer.

In some instances, at least one of the first solution, the first polymer component, the second solution, the second polymer component, and the dendrimer further includes one or more additives. Generally, the amount of additive may vary depending on the application, tissue type, concentration of the dendrimer in the second solution, the type of dendrimer, concentration of the second polymer component in the second solution, the type of second polymer component, the type of first polymer component, and/or the concentration of the first polymer component in the first solution. Example of suitable additives, include but are not limited to, pH modifiers, thickeners, antimicrobial agents, colorants, surfactants, and radio-opaque compounds. Specific examples of these types of additives are described herein. In one embodiment, at least one of the first solution, the first polymer component, the second solution, the second polymer component, and the dendrimer comprises a foaming additive.

In particular embodiments, at least one of the first solution, the first polymer component, the second solution, the second polymer component, and the dendrimer includes one or more drugs. In such embodiments, the hydrogel composites or compositions may serve as a matrix material for controlled delivery of the one or more drugs. The one or more drugs may be essentially any pharmaceutical agent suitable for local, regional, or systemic administration from a quantity of the hydrogel composite or composition that has been applied to one or more tissue sites in a patient. In one embodiment, the one or more drugs comprises a thrombogenic agent. Non-limiting examples of thrombogenic agents include thrombin, fibrinogen, homocysteine, estramustine, and combinations thereof. In another embodiment, the one or more drugs comprises an anti-inflammatory agent. Non-limiting examples of anti-inflammatory agents include indomethacin, salicyclic acid acetate, ibuprophen, sulindac, piroxicam, naproxen, and combinations thereof. In still another embodiment, the one or more drugs comprises an anti-neoplastic agent. In still other embodiments, the one or more drugs is one for gene therapy. For example, the one or more drugs may comprise siRNA molecules to combat cancer. In a particular embodiment, the one or more drugs comprises human bone morphogenetic protein 2. Other drugs are envisioned.

In other particular embodiments, at least one of the first solution, the first polymer component, the second solution, the second polymer component, and the dendrimer includes one or more cells. For example, in any of these embodiments, the hydrogel composites or compositions may serve as a matrix material for delivering cells to a tissue site at which the hydrogel composites or compositions have been applied. In embodiments, the cells may comprise endothelial cells (EC), endothelial progenitor cells (EPC), hematopoietic stem cells, or other stem cells. In one embodiment, the cells are capable of releasing factors to treat cardiovascular disease and/or to reduce restenosis. Other types of cells are envisioned.

### Formation of Hydro Gel Composites and Compositions

Generally, the hydrogel composites and compositions described herein may be formed by combining the first solution and the second solution in any manner. In some embodiments, the first solution, and the second solution are combined before contacting a biological tissue. In other embodiments, the first solution, and the second solution are combined, in any order, on or in a biological tissue. In further embodiments, the first solution is applied to a first biological tissue, the second solution is applied to a second biological tissue, and the first and second biological tissues are contacted. In still a further embodiment, the first solution is applied to a first region of a biological tissue, the second solution is applied to a second region of a biological tissue, and the first and second regions are contacted.

Generally, the hydrogel composites and compositions may be applied to a biological tissue as a drug delivery composition. The hydrogel composites and compositions also may be configured as a tissue adhesive or sealant.

The hydrogel composites and compositions may be applied to the biological tissue using any suitable tool and methods. Non-limiting examples include the use of syringes or spatulas. Double barrel syringes with rigid or flexible discharge tips, and optional extension tubes, known in the art are envisioned.

As used herein, the hydrogel composites and compositions are a “treatment” when they improve the response of at least one biological tissue to which they are applied. In some embodiments, the improved response is slowing or reversing tumor growth, inducing cytotoxicity in cancer cells, lessening overall inflammation, improving the specific response at the wound site/interface of the tissue and hydrogel composites or compositions, enhancing healing, repairing torn or broken tissue, or a combination thereof. As used herein, the phrase “lessening overall inflammation” refers to an improvement of histology scores that reflect the severity of inflammation. As used herein, the phrase “improving the specific response at the wound site/interface of the tissue and hydrogel composite or compositions” refers to an improvement of histology scores that reflect the severity of serosal neutrophils. As used herein, the phrase “enhancing healing” refers to an improvement of histology scores that reflect the severity of serosal fibrosis.

In embodiments, the hydrogel composites and compositions may be used in challenging or awkward implantation environments, including under flowing liquids and/or in inverted geometries.

Before or after contacting one or more biological tissues, the hydrogel composites and compositions may be allowed adequate time to cure or gel. When the hydrogel composites and compositions “cure” or “gel,” as those terms are used herein, it means that the one or more functional groups of the first polymer have undergone one or more reactions with the dendrimer and/or second polymer, and one or more biological tissues. Not wishing to be bound by any particular theory, it is believed that the hydrogel composites and compositions described herein are effective because the first polymer component reacts with both (i) the dendrimer and/or second polymer component, and (ii) the surface of the biological tissues. In certain embodiments, the first polymer component's aldehyde functional groups react with the amines on (i) the dendrimer and/or second polymer component, and (ii) the biological tissues to form imine bonds. In these embodiments, it is believed that the amines on the dendrimer and/or second polymer component react with a high percentage of the aldehydes of the first polymer component, thereby reducing toxicity and increasing biocompatibility of the hydrogel composites and compositions. Typically, the time needed to cure or gel the hydrogel composites and compositions will vary based on a number of factors, including, but not limited to, the characteristics of the first polymer component, second polymer component and/or dendrimer, the concentrations of the first solution and second solution, the pH of the first and second solution, and the characteristics of the one or more biological tissues. In embodiments, the hydrogel composites and compositions will cure sufficiently to provide desired bonding or sealing shortly after the components are combined. The gelation or cure time should provide that a mixture of the components can be delivered in fluid form to a target area before becoming too viscous or solidified and then once applied to the target area sets up rapidly thereafter. In one embodiment, the gelation or cure time is less than 120 seconds. In another embodiment, the gelation or cure time is between 3 and 60 seconds. In a particular embodiment, the gelation or cure time is between 5 and 30 seconds.

### Tissue Specific Formulations

Generally, the hydrogel composites and compositions may be adjusted in any manner to compensate for differences between tissues. In one embodiment, the amount of first polymer component is increased or decreased while the amount of dendrimer and/or second polymer component is unchanged. In another embodiment, the amount of dendrimer and/or second polymer component is increased or decreased while the amount of first polymer component is unchanged. In another embodiment, the concentration of the first polymer component in the first solution is increased or decreased while the second solution is unchanged. In yet another embodiment, the concentration of the dendrimer and/or second polymer component in the second solution is increased or decreased while the first solution is unchanged. In a further embodiment, the concentrations of the both the first polymer component in the first solution and the dendrimer and/or second polymer component in the second solution are changed.

When the amine density on the surface of a particular biological tissue is unknown due to disease, injury, or otherwise, an excess of the first solution may, in some embodiments, be added when the hydrogel composites and compositions are first applied, then the amount of first solution may be reduced, e.g., incrementally or drastically, until the desired effect is achieved. The “desired effect,” in this embodiment, may be an appropriate or adequate curing time, adhesion, sealing, treatment, drug delivery, or a combination thereof. Not wishing to be bound by any particular theory, it is believed that an excess of the first solution may be required, in some instances, to obtain the desired effect when the amine density on a biological tissue is low. Therefore, adding an excess will help the user, in this embodiment, achieve adequate sealing or adhesion or treatment in less time.

In other embodiments, however, a lower amount of the first solution may be added when the hydrogel composites and compositions are first applied, then the amount of first solution may be increased, e.g., incrementally or drastically, until the desired effect is achieved, which may be adequate curing time, adhesion, sealing, treatment, or a combination thereof.

In embodiments, the hydrogel composites and compositions can be optimized in view of a target biological tissue, by adjusting one or more of the following: rheology, mechanics, chemistry/adhesion, degradation rate, drug delivery, and bioactivity. These can be adjusted, in embodiments, by altering the type and/or concentration of the one or more hydrogel particles, the type and/or concentration of the first polymer component, and type and/or concentration of the dendrimer, the type and/or concentration of the second polymer component, or a combination thereof.

### Hydrogel Composite and Composition Kits

In another aspect, a kit is provided that comprises a first part that includes the first solution, and a second part that includes the second solution. The kit may further include an applicator or other device means, such as a multi-compartment syringe, for storing, combining, and delivering the two solutions and/or the resulting hydrogel composites and compositions to a tissue site.

In one embodiment, the kit comprises separate reservoirs for the first solution and the second solution. In certain embodiments, the kit comprises reservoirs for first solutions of different concentrations. In other embodiments, the kit comprises reservoirs for second solutions of different concentrations.

In one embodiment, the kit comprises instructions for selecting an appropriate concentration or amount of at least one of the first solution and/or second solution to compensate or account for at least one characteristic of one or more biological tissues. In one embodiment, the hydrogel composites and compositions are selected based on one or more predetermined tissue characteristics. For example, previous tests, may be performed to determine the number of density of bonding groups on a biological tissue in both healthy and diseased states. Alternatively, a rapid tissue test may be performed to assess the number or density of bonding groups. Quantification of tissue bonding groups can be performed by contacting a tissue with one or more materials that (1) have at least one functional group that specifically interacts with the bonding groups, and (2) can be assessed by way of fluorescence or detachment force required to separate the bonding groups and the material. In another embodiment, when the density of bonding groups on a biological tissue is unknown, an excess of the first polymer having one or more aldehydes, may be initially added as described herein to gauge the density of bonding groups on the surface of the biological tissue.

In certain embodiments, the kit comprises at least one syringe. In one embodiment, the syringe comprises separate reservoirs for the first solution and second solution. The syringe may also comprise a mixing tip that combines the two solutions as the plunger is depressed. The mixing tip may be release-ably securable to the syringe (to enable exchange of mixing tips), and the mixing tip may comprise a static mixer. In some embodiments, the reservoirs in the syringe may have different sizes or accommodate different volumes of solution. In other embodiments, the reservoirs in the syringe may be the same size or accommodate the same volumes of the solution.

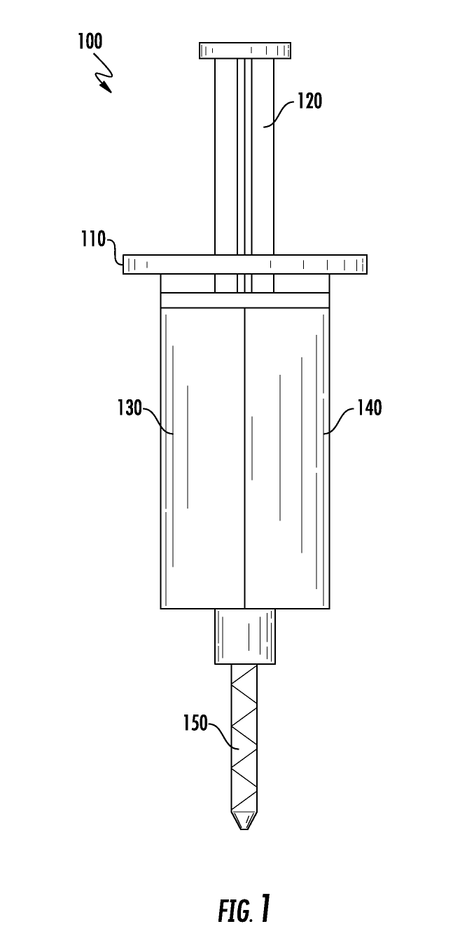

FIG. 1 depicts one embodiment of a syringe 100. The syringe 100 includes a body 110 with two reservoirs (130, 140). A first solution is disposed in the first reservoir 130, and a second solution is disposed in the second reservoir 140. The two reservoirs (130, 140) are emptied by depressing the plunger 120, which pushes the contents of the two reservoirs (130, 140) into the mixing tip 150 and out of the syringe 100.

In a further embodiment, one or more of the reservoirs of the syringe may be removable. In this embodiment, the removable reservoir may be replaced with a reservoir containing a first solution or second solution of a desired concentration.

In a preferred embodiment, the kit is sterile. For example, the components of the kit may be packaged together, for example in a tray, pouch, and/or box. The packaged kit may be sterilized using known techniques at suitable wavelengths (where applicable), such as electron beam irradiation, gamma irradiation, ethylene oxide sterilization, or other suitable techniques.

The phrases “C₁-C₂₀ hydrocarbyl,” “C₁-C₅ hydrocarbyl,” “C₂ hydrocarbyl,” and the like, as used herein, generally refer to aliphatic, aryl, or arylalkyl groups containing 1 to 20, 1 to 5, or 2 carbon atoms, respectively. Examples of aliphatic groups, in each instance, include, but are not limited to, an alkyl group, a cycloalkyl group, an alkenyl group, a cycloalkenyl group, an alkynyl group, an alkadienyl group, a cyclic group, and the like, and includes all substituted, unsubstituted, branched, and linear analogs or derivatives thereof, in each instance having 1 to about 20 carbon atoms, 1 to 5 carbon atoms, 2 carbon atoms, etc. Examples of alkyl groups include, but are not limited to, methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl. Examples of aryl or arylalkyl moieties include, but are not limited to, anthracenyl, azulenyl, biphenyl, fluorenyl, indan, indenyl, naphthyl, phenanthrenyl, phenyl, 1,2,3,4-tetrahydro-naphthalene, tolyl, xylyl, mesityl, benzyl, and the like, including any heteroatom substituted derivative thereof.

Unless otherwise indicated, the term “substituted,” when used to describe a chemical structure or moiety, refers to a derivative of that structure or moiety wherein one or more of its hydrogen atoms is substituted with a chemical moiety or functional group such as alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), tertiary amine (such as alkylamino, arylamino, arylalkylamino), aryl, aryloxy, azo, carbamoyl (—NHC(O)O— alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) or urea (—NHCONH-alkyl-).

In the descriptions provided herein, the terms “includes,” “is,” “containing,” “having,” and “comprises” are used in an open-ended fashion, and thus should be interpreted to mean “including, but not limited to.” When methods and composite materials are claimed or described in terms of “comprising” various components or steps, the composite materials and methods can also “consist essentially of” or “consist of” the various components or steps, unless stated otherwise.

The terms “a,” “an,” and “the” are intended to include plural alternatives, e.g., at least one. For instance, the disclosure of “a drug,” “a hydrogel particle,” “a crosslinker”, and the like, is meant to encompass one, a plurality of, or mixtures or combinations of more than one, drug, hydrogel particle, crosslinker, and the like, unless otherwise specified.

Various numerical ranges may be disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. Moreover, all numerical end points of ranges disclosed herein are approximate. As a representative example, Applicant discloses, in one embodiment, an average diameter of about 125 nm to about 145 nm. This range should be interpreted as encompassing an average diameter of about 125 nm to about 145 nm, and further encompasses “about” each of 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, and 144 nm, including any ranges and sub-ranges between any of these values.

### EXAMPLES

The present invention is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein.

### Example 1—Doxorubicin-Conjugated Polyaldehyde Hydrogel Particles

Dual sensitive hydrogel particles were fabricated by single-emulsion technique, and the hydrogel particle size was tuned by water/oil ratio, or polymer/crosslinker ratio. The hydrogel particles of this example were fabricated at a 1/5 water/oil ratio, and a 1/5 polymer/crosslinker ratio.

Polyaldehyde was synthesized by oxidizing dextran with sodium periodate. Linear dextran (18.9 g, 10 kDa) was dissolved in water with sodium periodate (17.6 g) for 5 h to create dextran aldehyde (50% oxidation of glucose rings, 2 aldehyde groups per oxidized glucose ring). The reaction mixture was dialyzed (MEMBRA-CEL Dialysis Tubing, molecular weight cutoff of 3500 Da, Viskase Companies, Inc.). A dry powder of oxidized dextran was obtained with lyophilization.

Doxorubicin (Dox), a chemo-therapeutic drug, was conjugated to the dextran aldehyde (Dex) to form a conjugate via reversible imine bond formation. Specifically, doxorubicin loaded dual sensitive hydrogel particles were fabricated by a single emulsion technique. Oxidized dextran (10 mg) and doxorubicin (2.9 mg) was dissolved in a polyvinyl alcohol (PVA) (5% w/v) aqueous solution, and triethylamine (0.7 ml) was added to form a dextran:doxorubicin (Dex:Dox) conjugate.

The disulfide bond-containing crosslinker cystamine dihydrochloride (1.126 mg) was dissolved in PVA solution (0.5 ml, 5% w/v). Both PVA solutions were mixed vigorously with chloroform (volume ratio 1:5) separately, and stirred for 6 h to create an emulsion. The two emulsions were mixed and stirred for 1 h to crosslink the polyaldehyde. Disulfide bond can be cleaved in the presence of the highly concentrated reductant present in cancer cells, such as glutathione (GSH) and dithiothreitol (DTT).

The disulfide bond of the cystamine crosslinker and the imine bond of the Dex:Dox conjugated conferred redox-pH dual sensitivity to the hydrogel particles of this example.

The aldehyde residue of the hydrogel particles of this example provided facile interaction with amine containing moieties, such as mono amino-PEG, dye, cell-penetrating peptides, or a combination thereof.

The hydrogel particles were collected by ultra-speed centrifuge, and washed 3 times with deionized (DI) water.

Drug-free hydrogel particles were fabricated in the same manner, but without doxorubicin.

Non-redox sensitive hydrogel particles also were prepared in the same manner, but cystamine dihydrochloride was replaced by hexamethylenediamine. Cystamine was replaced by hexasmethylenediamine to fabricate the non-redox sensitive nanogels that served as a control (NR NGs).

The success of Dex oxidization and Dex:Dox conjugate formation was confirmed by ¹H-NMR (300 MHz, DMSO-d₆).

It was noteworthy that the hydrogel particles of this example showed high drug loading efficiency irrespective of formulation, likely due to Dex:Dox conjugate formation. The loading efficiency was at least 90% when the dextran:crosslinker molar ratio was 1:1, 1:5, or 1:10.

The size of the hydrogel particles of this example was measured by Dynamic Light Scattering (DynaPro NanoStar), and the morphology of the hydrogel particles was observed by Transmission Electron Microscopy (FEI, Tecnai, Multipurpose TEM).

The average size (nm) of the hydrogel particles of this example was adjusted from 135.93±19.53 nm to 1112.16±193.40 nm, as confirmed by Dynamic Light Scattering (DLS) and depicted at the following table:

| | | Polymer to Crosslinker Ratio | | |
|---|---|---|---|---|
| Water to Oil Ratio | | 1:1 | 1:5 | 1:10 |
| 1:1 | Before | 623.55 ± 45.07 | 988.38 ± 64.85 | 1112.16 ± 193.40 |
| | After | 42.97 ± 9.29 | 58.29 ± 9.84 | 43.19 ± 6.48 |
| 1:5 | Before | 150.02 ± 13.00 | 135.93 ± 19.53 | 181.32 ± 21.32 |
| | After | 38.86 ± 8.54 | 24.69 ± 6.74 | 20.75 ± 6.71 |

As depicted in the foregoing table, hydrogel particles with different formulations were tested “Before” and “After” being treated with a GSH solution (10 mM).

The morphology of the hydrogel particles was observed by TEM. Spherical hydrogel particles were evenly distributed, and their size corroborated the DLS results.

The hydrogel particles of this example also had a cell toxicity comparable to poly (lactic-co-glycolic acid) nanoparticles at various dilutions, as determined by an MTT assay (FIG. 2), which was in accordance with a Live/Dead assay un which redox hydrogel nanoparticles were incubated with 3T3 fibroblasts for 1 day or 3 days, and MDA for 1 day or 3 days.

### Example 2—Compositions Including Doxorubicin-Conjugated Polyaldehyde Hydrogel Particles

The host hydrogel of this example was composed of oxidized dextran (Dex) and hydroxylated generation five (G5) dendrimer (Den). The crosslinking integrity of Dex:Den was derived from pH-sensitive imine bonds, that formed through a Schiff-base reaction between amines and aldehydes. The host hydrogel was prepared by a known technique (see N. Artzi, et al., Adv. Mater., 2009, 21, 3399).

Composite hydrogels were fabricated by mixing a solution of the hydrogel particles of Example 1 with oxidized dextran solution before gelation with Den solution. With both the hydrogel particles and dextran labeled with fluorescent dyes, fluorescent imaging of composite hydrogels revealed that the hydrogel particles (labeled with Alexa Fluor 647) of Example 1 were evenly fused into the skeleton of the Dex:Den hydrogels. The dextran was labeled with FITC.

The presence of sulfur in the compositions was confirmed by Energy Dispersive X-ray assisted Scanning Electron Microscopy (EDX-SEM). The EDX-calculated theoretical atomic percentage of each element in the host hydrogel with and without the hydrogel particles is depicted at the following table:

| Sample | Element | EDX (%) | Theoretical (%) |
|---|---|---|---|
| Host Hydrogel | C | 51.29 | 58.33 |
| with Hydrogel | O | 20.01 | 30.27 |
| Particles | N | 28.18 | 10.70 |
| | S | 0.52 | 0.70 |
| Host Hydrogel | C | 50.29 | 58.57 |
| | O | 31.33 | 31.15 |
| | N | 18.38 | 10.28 |

### Example 3—In Vitro Drug Release

A triple-negative breast cancer cell line, MDA-MB-231 (MDA), was chosen as a tumor model for the in vitro studies of Example 3, and for in vivo studies of Example 4.

In vitro drug release behavior of both hydrogel particles and composite hydrogels was studied using different solutions simulating the physiological conditions in healthy and cancer cells. Saline solution with pH 7.4 was used to mimic a healthy tissue microenvironment. To mimic tumor tissue, the pH was set at 6.5 and 5, and a redox environment was created by adding GSH (10 mM).

The Dex:Dox conjugate of Example 1 or a solution (1 ml) of the hydrogel particles of Example 1 was placed in a Dialysis Membranes with 500-1000 molecular weight cut off (Spectrum Laboratories), then immersed into selected releasing media (100 ml). The concentration of doxorubicin in releasing media was determined by measuring UV absorbance at 480 nm by Microplate Reader (Varioskan Flash Multimode Reader, Thermo Scientific).

To assess the release of hydrogel particles from composite hydrogels, the hydrogel particles of Example 1 were conjugated with Alexa Fluor® 647 (Invitrogen) prior to composite hydrogel fabrication.

The composite hydrogels of Example 2 were placed in 48-wells plates, and immersed with releasing media (0.5 ml). Releasing media was replaced at a predetermined time point, and the fluorescence of the composite hydrogels was measured by Microplate Reader. All releasing media were PBS based. An HCl solution (1 M) was used to adjust the pH to 6.5 or 5. GSH was added for a concentration of 10 mM to make a redox releasing media.

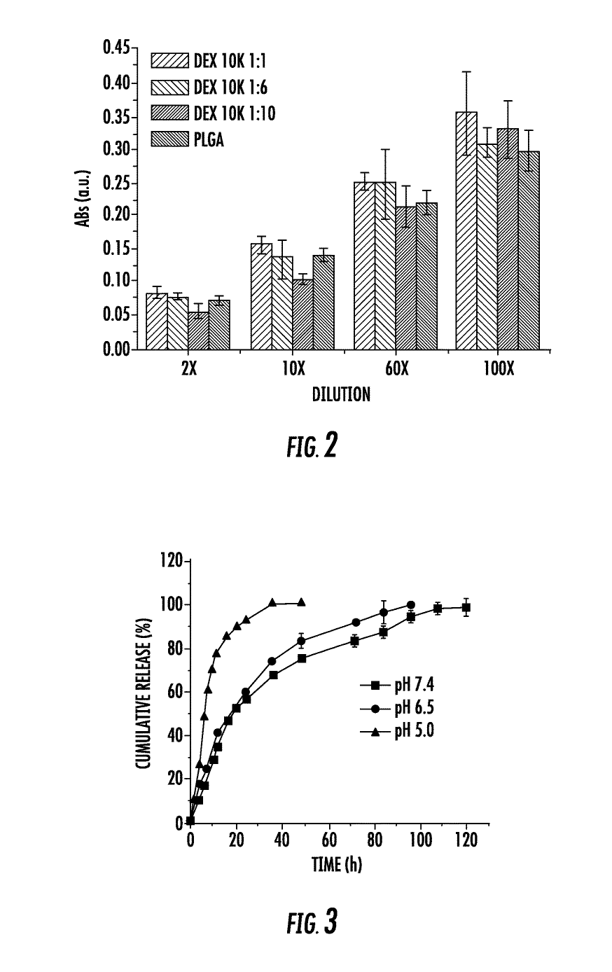

As imine bond hydrolysis is enhanced under acidic condition, the Dex-Dox conjugate exhibited an accelerated release under both pH 6.5 and 5, as depicted at FIG. 3.

As shown in FIG. 4, the presence of GSH (10 mM) accelerated Dox release from the hydrogel particles to 4 d instead of 7 d. This result confirmed the redox sensitivity of the hydrogel particles of Example 1.

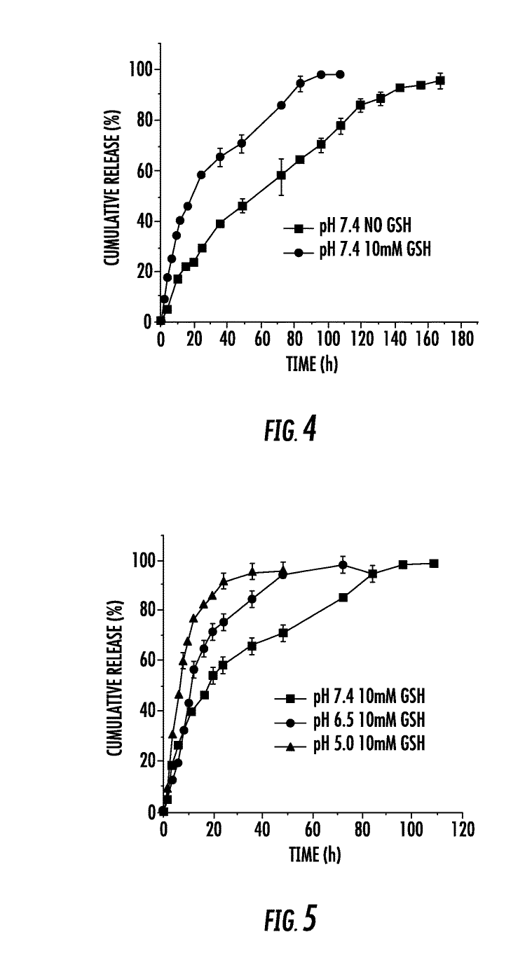

The hydrogel particles of Example 1 also were placed in 10 mM GSH media at various pHs. The release of Dox from dual-sensitive hydrogel particles at different pHs in the presence of GSH showed that acidic conditions (both pH 5 and pH 6.5) were able to facilitate Dox release. As depicted at FIG. 5, 90% of Dox was released within 24 h under a cancer cell-mimicking environment (pH 5 and 10 mM GSH).

Release of hydrogel particles from a host hydrogel also was measured. The data at FIG. 6 revealed that acidic (pH 5) and redox (10 mM GSH) conditions reduced the release time from 20 d to 13 d.

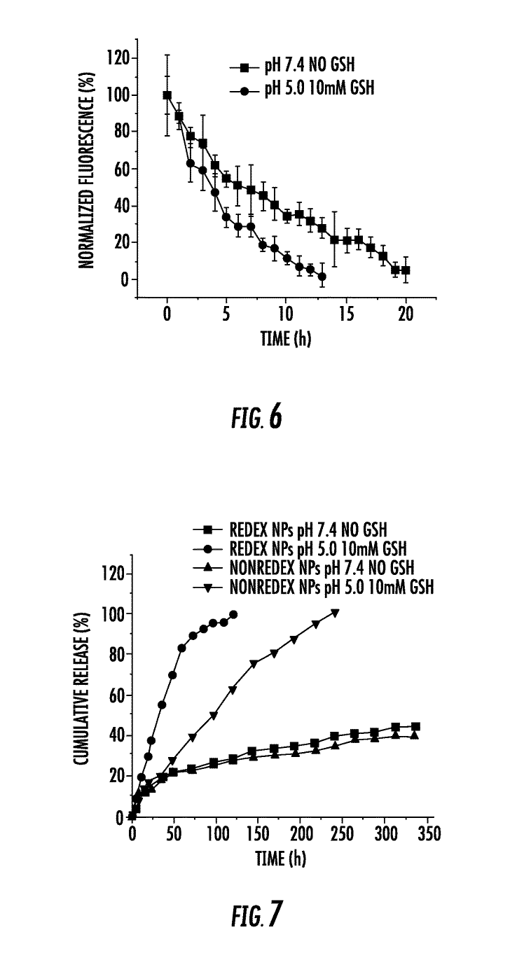

It is noteworthy that with the acidic and redox conditions inherent to the tumor milieu, the compositions were still able to provide a sustained release of hydrogel particles. For comparison, non-redox sensitive hydrogel particles were embedded into a host hydrogel in the same manner as the doxorubicin:dextran conjugates of Example 1. As shown at FIG. 7, hydrogels with both types of hydrogel particles presented substantially the same release profile in a healthy cell environment (pH 7.4 and no GSH). Conversely, both compositions showed accelerated release of Dox under cancer cell-mimicking conditions (pH 5 and 10 mM GSH) due to the pH sensitivity of both types of hydrogel particles. However, the host hydrogels containing redox sensitive hydrogel particles showed a much quicker release of Dox (5 d) than the host hydrogels that contained non-redox sensitive hydrogel particles (10 d).

This result demonstrated the dual sensitivity of the hydrogel particles of Example 1. The in vitro drug release studies of this example provided evidence that the hydrogel hosts were able to offer a pH regulated and sustained release of hydrogel particles, while the hydrogel particles delivery drug while inside the cells under acidic and redox conditions, thereby possibly providing rapid anti-cancer therapy.

### Example 4—Cellular Viability, Uptake, and Selectivity Studies

To better predict the therapeutic efficacy of the hydrogel particles in vitro, cellular viability, uptake and selectivity studies were performed.

MDA-MB-231 cells (from triple negative breast cancer) and 3T3 human fibroblasts were grown in Dulbecco's modified Eagle's medium (DMEM, Invitrogen) supplemented with glutamine (4 mM), 10% heat inactivated fetal bovine serum (Gibco), penicillin (100 U/ml) and streptomycin (100 μg/ml) (Invitrogen) and maintained at 37° C. in 5% CO₂. Both 3T3 fibroblasts and MDA-MB-231 cells were seeded at a density of 1×10⁵ cells/well in 24-well plates and grown for 24 h prior to incubation of dual-sensitive nanogels. On the day of incubation, the cells were approximately 50% confluent. For the therapeutic efficacy study, cells were incubated with Dox-loaded dual sensitive hydrogel particles (10 mg/ml). Cell viability was evaluated after 1 d and 3 d of incubation. For confocal microscopy, cells were fixed with paraformaldehyde (4%) in PBS for 15 min at 37° C. and mounted in ProLong® Gold Antifade Reagent with DAPI (Invitrogen) to allow for nuclear staining. Images of cells were taken with a Nikon AIR Ultra-Fast Spectral Scanning Confocal Microscope. For flow cytometry, both cells were incubated with nanogels (0.2 mg/mL) were analysed and data were acquired on FACS LSR Fortessa HTS-1 (BD Biosciences) flow cytometer.

Significantly reduced cell number was observed for cancer cells (MDA) after 1 d of incubation, as shown at FIG. 8. Meanwhile, healthy 3T3 human fibroblasts showed a significantly reduced cell number as well after 3 d of incubation. This result complied with the in vitro drug release studies, and further demonstrated the selectivity of the Dox:Dex conjugates of Example 1.

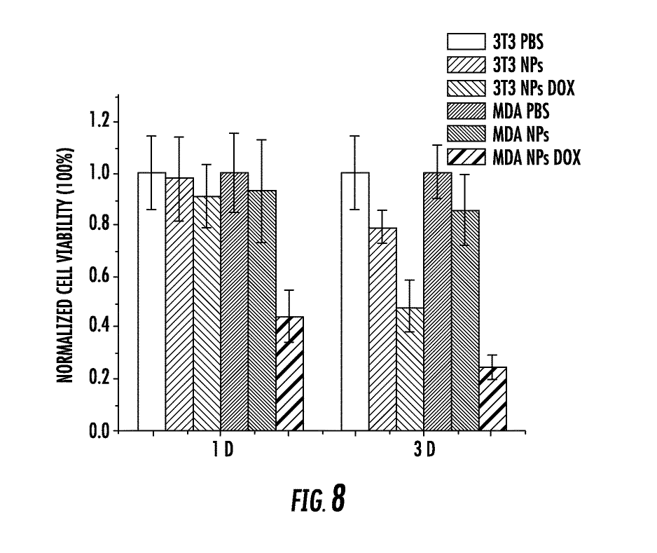

Although the hydrogel composition may be locally implanted over tumor tissue, the released hydrogel particles may, in some instances, have a chance of being uptaken by one or more surrounding healthy tissues. Therefore, a cell-penetrating peptide —RGD- was conjugated onto the surface of the hydrogel particles. The RGD peptide conferred selective uptake by cancer cells, as depicted at FIG. 9A and FIG. 9B. The data revealed that the conjugation of RGD to the hydrogel particles successfully improved the selective uptake of the hydrogel particles by MDA cells (FIG. 9A), while no difference was shown for 3T3 fibroblasts (FIG. 9B).

This conclusion was corroborated with confocal microscopy images of the in vitro cellular uptake. In culture with 3T3 fibroblasts, a limited amount of hydrogel particles were internalized. However, a significantly larger quantity of hydrogel particles was uptaken by the cancer cells (MDA). A strong signal from the hydrogel particles was presented over the entire cytoplasm and perinuclear regions of the cells, which indicated cellular uptake rather than surface anchoring. Therefore, these in vitro cell culture studies demonstrated that the hydrogel nanoparticles had high selectivity and cellular uptake to cancer cells.

### Example 5—In Vivo Drug Release

An orthotopic triple-negative breast cancer was developed in female immunodeficient SCID hairless outbred (SHO, Crl:SHO-Prkdc^{scid}Hr^{hr}, 6 weeks) mice. To address non-invasive and real-time monitoring, mice were injected with luciferin before imaging to assess tumor burden, and all hydrogel particles were labeled with Alexa-fluor® 647 prior to administration.

Orthotopic triple negative breast cancer mice model was developed for in vivo animal study. Tumors in the mammary fat pad were induced in female immunodeficient SCID hairless outbred (SHO) mice by injection of 5×10⁶ MDA-MB-231 cells stably expressing firefly luciferase, suspended in HBBS (50 μL) (Lonza) solution. For determination of tumor growth, individual tumors were measured using caliper and tumor volume was calculated by: Tumor volume (mm³)=width×(length²)/2. Treatments began when tumor volume reached about 100 mm³. Briefly, nanogels were administered both systemically and locally. For systemic administration mice were injected with nanogels (3 mg/mL) via tail-vein and the tumor growth was followed during 11 d. For local administration, pre-cured disks of hydrogel scaffold embedded with nanogels were formed and implanted subcutaneously on top of the fat mammary tumor in SCID mice. For analysis of tumor growth, non-invasive longitudinal monitoring of tumor progression was followed by scanning mice with the IVIS Spectrum-bioluminescent and fluorescent imaging system (Xenogen XPM-2 Corporation) from mice bearing mammary tumors from MDA-MB-231 cells (n=5 animals per treated group). 15 minutes before imaging, mice were intraperitoneally injected with D-luciferin (150 μL, 30 mg/mL, Perkin Elmer) in DPBS (Lonza). Whole-animal imaging was performed during 11 days. At the end of 11 d, mice were sacrificed and the organs harvested and imaged. All experimental protocols were in compliance with NIH guidelines for animal use.

Both intravenous administration (systemic) of hydrogel particles and on-site (orthotopic tumor) implantation of composite hydrogels (local) were conducted. For systemic administration, Dox-loaded dual sensitive hydrogel nanoparticles (DS NGs Dox) were compared to drug-free DS NGs, and Dox-loaded non-redox sensitive hydrogel particles (NR NGs Dox), with saline as negative control. For local administration, biosensitive composite hydrogels (Hyd with DS NGs Dox) were compared to free drug doped on the hydrogel (Hyd with DS NGs), composite hydrogels with non-redox sensitive hydrogel particles (Hyd with NR NGs Dox), and hydrogels formed directly by Dex:Dox conjugates (Hyd with Dox).

The study of biodistribution showed that hydrogel particles that were systemically administered accumulated at the liver and spleen other than the tumor site. The enlarged spleen (i.e. splenomegaly) suggested the toxicity caused by systemic administration, when compared to local administration.

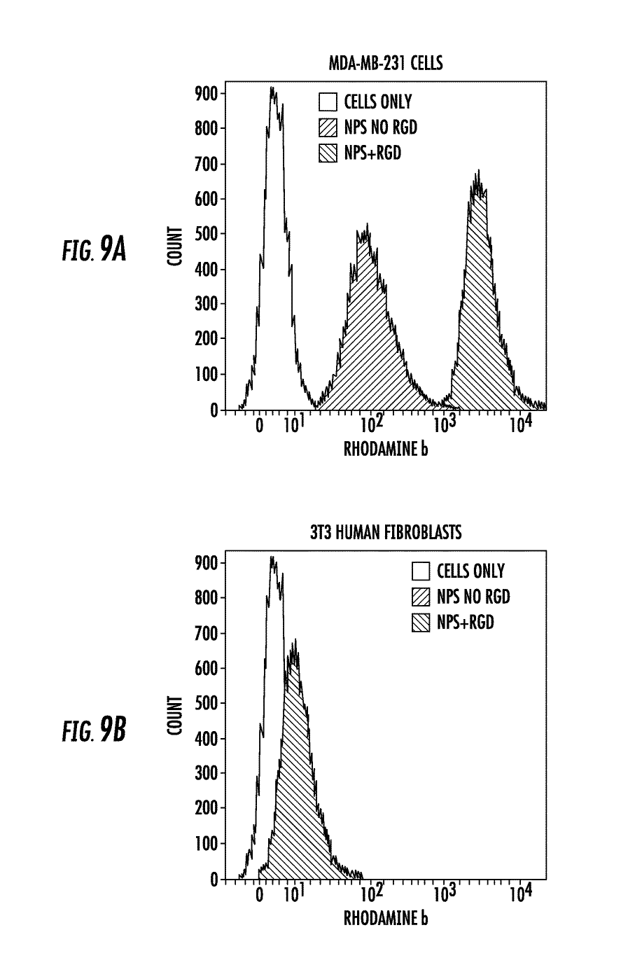

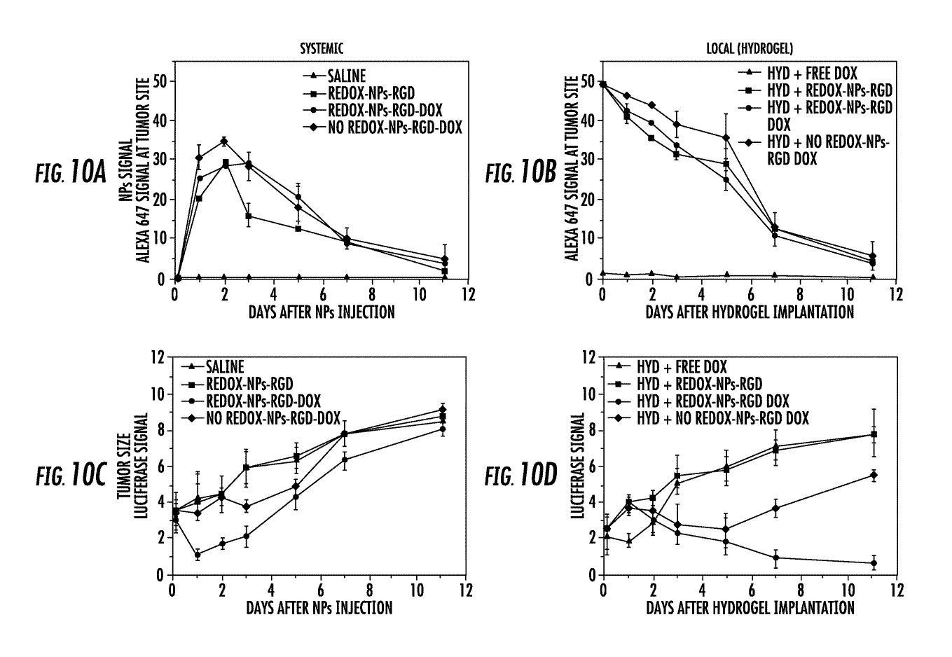

However, this issue was completely resolved by local administration of the therapeutic cargos (see, FIG. 10A, FIG. 10B, FIG. 10C, and FIG. 10D). The data at FIG. 10C and FIG. 10D showed that groups with drug-free devices (both hydrogel particles (FIG. 10C) and composite hydrogels (FIG. 10D)) had the same tumor size as the negative control at all time points. This indicated that Dox was solely responsible for anti-cancer therapeutic efficacy.

At 48 h following systemic treatment, hydrogel particles accumulated at the tumor site in all groups. No increase in tumor size was observed in the group of NR NGs Dox during the first 72 h (FIG. 10C). However, a significant reduction of tumor size after 24 h and slow tumor growth up to 72 h were observed in the group of DS NGs Dox. This showed that DS NGs conferred a more rapid and efficient treatment than NR NGs due to the rapid drug release of DS NGs in the tumor microenvironment, which complied with the results of the in vitro drug release studies of Example 3. Although a promising result was obtained with systemic administration of DS NGs, tumor growth started to dominate after 72 h.

Similar results were observed in groups treated with Hyd with Dox. Tumor shrinkage after 24 h and restrained tumor growth up to 48 h was observed, as shown at FIG. 10D. Nevertheless, hydrogels directly loaded with small therapeutics failed to afford long-term treatment, even with covalent conjugation. For groups with local administration (FIG. 10B), the profile of hydrogel particle release stayed substantially the same for all treated groups. No signal from the hydrogel particles was observed after 11 d, which was similar to the result observed for the in vitro release of dual-sensitive nanogels from composite hydrogels under conditions that mimicked healthy and cancerous environments.

Comparing groups of Hyd with DS NGs Dox and Hyd with NR NGs Dox, tumor shrinkage was observed in both groups up to 5 d, as shown at FIG. 10D. Although with no statistically significant difference, more shrinkage was shown in the group of Hyd with DS NGs Dox. However, the tumor recurred in the Hyd group with NR NGs Dox after 5 d, while the tumor kept shrinking up to 11 d in the group treated with Hyd with DS NGs Dox.

Moreover, an extensive reduction in vascularization in the tumor tissue was revealed only in the group treated with Hyd with DS NGs Dox. Both results indicated that sustained and efficient therapeutic effect was implemented by Hyd with DS NGs Dox.

We claim:

1. A composition comprising:
a hydrogel particle comprising—
(i) a crosslinked polymer comprising a crosslinker, and
(ii) a drug conjugated to the crosslinked polymer by a covalent bond that is pH sensitive or redox sensitive;
wherein the crosslinker comprises a moiety that is (1) redox sensitive when the covalent bond is pH sensitive, or (2) pH sensitive when the covalent bond is redox sensitive.

2. The composition of claim 1, wherein the moiety that is redox sensitive comprises a disulfide moiety.

3. The composition of claim 1, wherein the moiety that is pH sensitive comprises an imine moiety.

4. The composition of claim 1, wherein the covalent bond is pH sensitive, and the crosslinker comprises the moiety that is redox sensitive.

5. The composition of claim 4, wherein the covalent bond comprises an imine bond.

6. The composition of claim 4, wherein the crosslinker comprises a compound of the following formula: 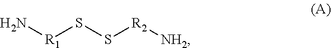
wherein R₁ and R₂ are independently selected from a divalent C₁-C₂₀ hydrocarbyl.

7. The composition of claim 6, wherein R₁ and R₂ are unsubstituted divalent C₁-C₅ hydrocarbyls, and the crosslinker has the following structure:

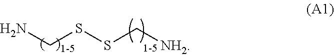

8. The composition of claim 6, wherein the crosslinker comprises a compound of formula (A), wherein R₁ and R₂ are unsubstituted divalent C₂ hydrocarbyls, and the crosslinker comprises cystamine.

9. The composition of claim 1, wherein the drug comprises one or more anti-tumor agents, one or more anti-angiogenic agents, or a combination thereof.

10. The composition of claim 1, wherein the drug comprises doxorubicin.

11. The composition of claim 1, wherein the hydrogel particle has an average diameter of about 50 nm to about 1,500 nm.

12. The composition of claim 1, wherein the hydrogel particle has an average diameter of about 100 nm to about 1,300 nm.

13. The composition of claim 1, further comprising a cell penetrating peptide conjugated to the crosslinked polymer.

14. The composition of claim 13, wherein the cell penetrating peptide includes RGD.

15. The composition of claim 1, wherein the crosslinked polymer comprises a polysaccharide having one or more aldehydes.

16. The composition of claim 1, further comprising a host hydrogel in which the hydrogel particle is dispersed.

17. A method of delivery of a drug to biological tissue, the method comprising:
providing a first solution comprising a first polymer component comprising a first polymer having one or more aldehydes;
providing a second solution comprising at least one of (i) a dendrimer comprising at least two branches with one or more surface groups, wherein about 25% to 100% of the surface groups comprise at least one primary or secondary amine, and (ii) a second polymer component comprising a second polymer having one or more amines;
combining the first and second solutions together to produce a hydrogel composite; and
contacting one or more biological tissues with the hydrogel composite,
wherein at least one of the first solution and the second solution comprises the composition of claim 1.

18. The method of claim 17, wherein the composition of claim 1 is substantially evenly dispersed in the first solution, the second solution, or both the first solution and the second solution.

19. The method of claim 17, wherein the concentration of the composition of claim 1 in at least one of the first solution and the second solution is about 0.01% to about 30% by weight of the first solution or the second solution, respectively.

20. The method of claim 19, wherein the concentration of the first polymer component in the first solution is about 0.01 percent to about 30 percent, by weight of the first solution.

21. The method of claim 17, wherein the first polymer has a number average molecular weight of about 1,000 Da to about 1,000,000 Da.

22. The method of claim 17, wherein the first polymer has a degree of functionalization of about 10% to about 75%.

23. The method of claim 17, wherein the first polymer comprises a nonionic polysaccharide.

24. The method of claim 23, wherein the nonionic polysaccharide comprises dextran, dextrin, cellulose, derivatives thereof, or a combination thereof.

25. The method of claim 17, wherein the first polymer comprises an anionic polysaccharide.

26. The method of claim 25, wherein the anionic polysaccharide comprises hyaluronic acid, chondroitin sulfate, alginate, cellulose gum, or a combination thereof.

27. The method of claim 17, wherein the first polymer comprises a cationic polysaccharide.

28. The method of claim 27, wherein the cationic polysaccharide comprises chitosan, cationic guar gum, cationic hydroxyethylcellulose, a polysaccharide modified with trimethylammonium groups, or a combination thereof.

29. The method of claim 17, wherein the first polymer comprises a hydrophilic polymer.

30. The method of claim 29, wherein the hydrophilic polymer is modified with polyester groups.

31. The method of claim 29, wherein the hydrophilic polymer comprises poly(vinyl alcohol), poly(acrylic acid), poly(acrylamide), poly(ethylene oxide), or a combination thereof.

32. The method of claim 17, wherein the first polymer comprises a hydrophobic polymer.

33. The method of claim 32, wherein the hydrophobic polymer comprises pendant hydrophilic polymers.

34. The method of claim 32, wherein the hydrophobic polymer comprises polycaprolactam, poly(lactic acid), polycaprolactone, or a combination thereof.

35. The method of claim 17, wherein the dendrimer component is present in the second solution at a concentration of about 0.01% to about 30%, by weight of the second solution.

36. The method of claim 17, wherein the dendrimer comprises a PAMAM dendrimer.

37. The method of claim 17, wherein the second polymer component is present in the second solution at a concentration of about 0.01% to about 30% by weight of the second solution.

38. The method of claim 17, wherein the total concentration of the second polymer component and the dendrimer in the second solution is about 0.01% to about 40% by weight of the second solution.

39. The method of claim 17, wherein the second polymer is a polyamine.

40. The method of claim 39, wherein the polyamine comprises amine-terminated multi-arm poly(ethylene oxide), polyethyleneimine, or a combination thereof.

41. The method of claim 17, wherein the second polymer comprises chitosan, collagen, gelatin, or a combination thereof.

42. The method of claim 17, wherein the first solution and the second solution are combined on a biological tissue.

43. The method of claim 17, wherein the first solution and the second solution are combined in a syringe.

44. The method of claim 17, wherein the second solution is applied to a tissue followed by the first solution.

45. The method of claim 17, wherein the one or more biological tissues comprise human tissue.

46. A kit for making a hydrogel composite, the kit comprising:
a first part that includes a first solution comprising a first polymer component comprising a first polymer having one or more aldehydes; and
a second part that includes a second solution comprising at least one of (i) a dendrimer comprising at least two branches with one or more surface groups, wherein about 25% to 100% of the surface groups comprise at least one primary or secondary amine, and (ii) a second polymer component comprising a second polymer having one or more amines,
wherein at least one of the first solution and the second solution comprises the composition of claim 1.

47. The kit of claim 46, further comprising a syringe, wherein the first solution and the second solution are stored in the syringe.

48. The kit of claim 47, wherein the syringe comprises separate reservoirs for the first solution and the second solution.

49. The kit of claim 48, wherein the syringe comprises a mixing tip.

50. A method for local delivery of a drug to a biological tissue, comprising:
applying to the biological tissue the composition of claim 16; and
permitting the hydrogel particle to diffuse from the composition into the biological tissue.

51. A method of treating cancer in a patient, comprising:
administering to the patient an effective amount of the composition of claim 16.

52. The method of claim 51, wherein the administering comprises applying the composition locally to one or more tumors in the patient's body.
